(19) United States
(12) Patent Application Publication
(10) Pub. No.: US 2022/0409141 A1
(43) Pub. Date: Dec. 29, 2022

(54) HUMAN BODY SENSING MAT

(71) Applicant: LG ELECTRONICS INC., Seoul (KR)

(72) Inventors: Homin LEE, Seoul (KR); Younjae LEE, Seoul (KR); Kihyuk LEE, Seoul (KR); Hyungyu PARK, Seoul (KR); Hyounggil YOON, Seoul (KR); Seonghyok KIM, Seoul (KR); Hyunoh KANG, Seoul (KR); Hyung YOON, Seoul (KR)

(73) Assignee: LG ELECTRONICS INC., Seoul (KR)

(21) Appl. No.: 17/780,773

(22) PCT Filed: Nov. 29, 2019
(86) PCT No.: PCT/KR2019/016693

§ 371 Date: May 27, 2022

(51) Int. Cl. A61B 5/00; A61B 5/273; A61B 5/01; A61B 5/11

(52) U.S. Cl. CPC A61B 5/6892; A61B 5/273; A61B 5/01; A61B 5/1116; A61B 5/4806; A61B 2562/063; A61B 2562/182

(57) ABSTRACT

A human body sensing mat for sensing the movement of a human body, comprises: a substrate; a sensor array including a plurality of fiber sensors for generating signals according to a distance to a specific object, and disposed on the substrate; and a driving unit for applying a voltage to the sensor array, wherein the sensor array includes a plurality of sensing lines arranged in parallel in a first direction, and regarding the plurality of sensing lines, a first distance between neighboring sensing lines connected to different electrodes is greater than a second distance between neighboring sensing lines connected to the same electrode. The human body sensing mat can analyze user's movements and biometric signals regardless of the user's posture.

### TECHNICAL FIELD

The present disclosure relates to a human body sensing mat that may analyze a motion and a bio-signal of a user.

### BACKGROUND ART

Sleep is one of important factors for physical and mental health of a human. Adequate sleep is effective in recovering from fatigue, improving immunity and concentration, relieving stress, reducing inflammation, restoring muscles, and the like.

Accordingly, sleep assisting devices, sleep inducing devices, or sleep analyzing devices for proper and effective sleep have emerged. However, most of the current devices may operate only when various sensors and the like are worn on a body part of a user.

Such devices may rather cause inconvenience to the user in getting proper sleep, and may be cumbersome to use such devices. Accordingly, there is an increasing demand for a device capable of analyzing a motion and a bio-signal of the user without being worn on the user's body.

As shown in FIG. 1, in the related art, the motion and the bio-signal of the user were analyzed by disposing a sensor 110 beneath the bedding. However, because the sensor 110 as shown in FIG. 1 is highly dependent on a sleeping posture of the user, there is a problem in that it is difficult to analyze the sleep of the user when the user sleeps in a specific posture. Specifically, as shown in FIG. 1, when the user sleeps in a first posture 10, the sensor 110 is capable of accurately analyzing the sleep. However, when the user sleeps in a second posture 21 as shown in FIG. 2, it becomes difficult for the sensor 110 to accurately analyze the sleep.

### DISCLOSURE

### Technical Problem

The present disclosure is to provide a human body sensing mat that may analyze a motion and a bio-signal of a user regardless of a posture of the user.

### Technical Solutions

A human body sensing mat according to an embodiment of the present disclosure includes a substrate, a sensor array including a plurality of fiber sensors for respectively generating signals based on a distance from a specific object, wherein the sensor array is disposed on the substrate, and a driver for applying a voltage to the sensor array, the sensor array includes a plurality of sensing lines arranged in parallel with each other in a first direction, and the plurality of sensing lines have a first spacing between adjacent sensing lines connected to different electrodes greater than a second spacing between adjacent sensing lines connected to the same electrode in a second direction.

The first spacing and the second spacing may be defined in a direction perpendicular to the first direction.

The human body sensing mat may further include a temperature sensor disposed on the substrate so as to measure a body temperature of a user, the sensor array may include a first sensor array disposed at one side of the substrate to correspond to an upper body of the user and a second sensor array disposed at the other side of the substrate to correspond to a lower body of the user, and the temperature sensor may be disposed between the first sensor array and the second sensor array.

The plurality of sensing lines may include a first sensing line and a second sensing line connected to different electrodes, the first sensing line and the second sensing line may be spaced apart from each other at a predetermined spacing and be arranged on the substrate in a zigzag form, the first spacing may be a distance between the first sensing line and the second sensing line closest to the first sensing line, and the second spacing may be a distance between a portion and another portion of the first sensing line or the second sensing line.

The first spacing may be in a range from 25 mm to 30 mm and a second spacing may be in a range from 10 mm to 15 mm.

The human body sensing mat may further include a plurality of connecting electrodes disposed at an edge of the substrate, wherein each of the plurality of connecting electrodes electrically connects each of the fiber sensors to the driver.

The human body sensing mat may further include a shielding layer for covering each of the connecting electrodes to block an electromagnetic field.

The shielding layer may be formed at an edge of the substrate.

The driver may output sleep information of a user using the signals respectively generated from the fiber sensors.

Each signal may be an impedance change caused by a distance between the user and each fiber sensor.

The driver may calculate posture information of the user based on the signals respectively generated from the fiber sensors and output the sleep information using the signals respectively generated from the fiber sensors and the posture information.

The driver may assign weights to the signals respectively generated from the fiber sensors based on the posture information and output the sleep information using the weighted signals.

### Advantageous Effects

According to the present disclosure, because the fiber sensors are individually arranged at the central portion of the mat, the breathing, tossing and turning, and the like of the user may be sensed regardless of the posture of the user. Therefore, the present disclosure may analyze the motion and the bio-signal of the user regardless of the posture of the user.

In addition, according to the present disclosure, because the different signals are generated depending on the position of the mat, the sleeping posture of the user may be recognized. The present disclosure may improve the sleep analysis accuracy by utilizing the sleeping posture information of the user.

### DESCRIPTION OF DRAWINGS

FIGS. 1 and 2 are conceptual views showing a conventional human body sensing mat.

FIG. 3 is a conceptual diagram showing a human body sensing mat according to the present disclosure.

FIG. 4 is a cross-sectional view of a human body sensing mat according to the present disclosure.

FIG. 5 is a conceptual diagram illustrating a principle of a LC-Tank.

FIG. 6 is a block diagram illustrating a driver according to the present disclosure.

FIGS. 7 and 8 are cross-sectional views of a mat having a shield.

FIG. 9 is a conceptual diagram illustrating an example of application of a human body sensing mat according to the present disclosure.

FIG. 10 is a graph showing a result of synthesizing signals generated from fiber sensors.

FIG. 11 is a diagram conceptually illustrating arrangement of sensor arrays of a human body sensing mat according to the present disclosure.

FIG. 12 is an enlarged view of A in FIG. 11.

FIG. 13 is a diagram showing a result of sensing, by a human body sensing mat in FIG. 11, breathing of a user.

FIG. 14 is a diagram illustrating a table comparing measurement results of PSG equipment used in a general hospital and a human body sensing mat according to the present disclosure.

### BEST MODE

Hereinafter, an embodiment disclosed herein will be described in detail with reference to the accompanying drawings. Regardless of the reference numerals, the same or similar components will be assigned the same reference numerals, and duplicated descriptions thereof will be omitted. In describing the embodiment disclosed herein, when it is determined that a detailed description of a related known technology may obscure the gist of the embodiment disclosed herein, a detailed description thereof will be omitted. In addition, the accompanying drawings are only for easy understanding of the embodiment disclosed herein. It should be understood that the technical idea disclosed herein is not limited by the accompanying drawings and includes all changes, equivalents, or substitutes included in the spirit and scope of the present disclosure.

Hereinafter, a human body sensing mat according to an embodiment of the present disclosure will be described with reference to the accompanying drawings. Herein, an example of analyzing sleep of a user using the human body sensing mat is described, but the human body sensing mat according to the present disclosure may not be limited to the sleep of the user, and may analyze a motion and a bio-signal of the user from various viewpoints.

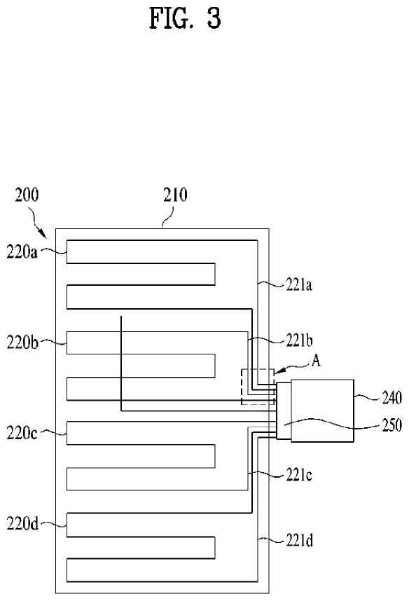

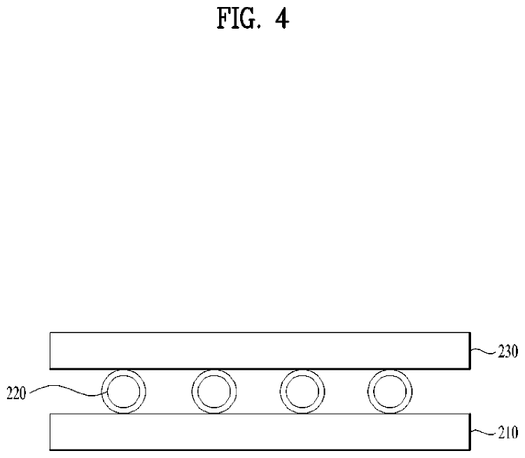

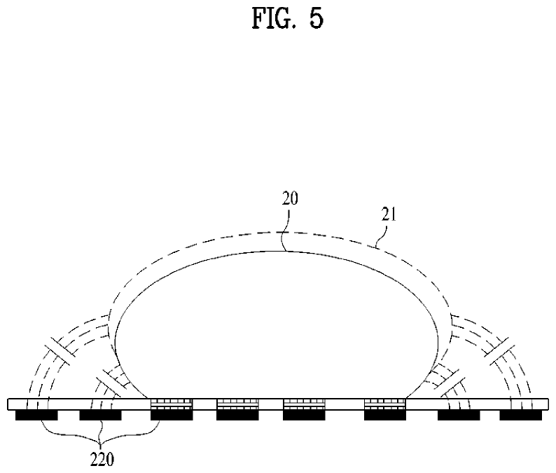

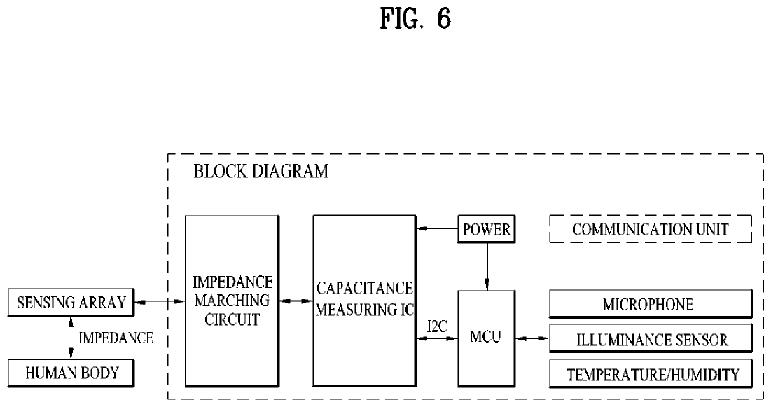

FIG. 3 is a conceptual diagram showing a human body sensing mat according to the present disclosure, FIG. 4 is a cross-sectional view of a human body sensing mat according to the present disclosure, FIG. 5 is a conceptual diagram illustrating a principle of a LC-Tank, and FIG. 6 is a block diagram illustrating a driver according to the present disclosure.

The human body detection sensor 200 according to the present disclosure includes a substrate 210, sensor arrays 220a to 220d, a shield layer 230, and a driver 240. However, the present disclosure may not be limited thereto, and the human body detection sensor according to the present disclosure may include components of the number greater or smaller than the number of components described above.

Hereinafter, the above-described components will be described in detail.

The substrate 210 is used as means for fixing the sensor array 220a to 220d and the shield layer 230. Because the mat according to the present disclosure may be used in different aspects, such as being used in a fully unfolded state, being used in a partially folded state, or the like, the substrate 210 is preferably made of a bendable and flexible material.

The material constituting the substrate 210 is not limited, but the substrate 210 is preferably made of a material that is bendable and washable because of a nature of the mat.

The sensor arrays 220a to 220d are disposed on the substrate 210. The sensor arrays 220a to 220d include a plurality of fiber sensors 220a to 220d that generate signals based on a distance from a specific object. In this regard, each of the fiber sensors may be composed of an electrode whose impedance value changes based on the distance from the specific object.

Specifically, each of the fiber sensors 220a to 220d may be formed as an LC Tank. The LC tank is a sensor that infers a change in a capacitance using an amount of oscillation frequency shifted based on the change in the capacitance. When the LC Tank is used, a distance between a user's body and each of the fiber sensors may be sensed.

The change in the impedance in each of the fiber sensors 220a to 220d may be measured, and a posture and a breathing pattern of the user may be calculated using the measurement.

For example, as shown in FIG. 5, an impedance value of the fiber sensor 220 of when the user is lying in a first state 20 on the mat according to the present disclosure and an impedance value of the fiber sensor 220 of when a state of the user becomes a second state 21 via breathing are different from each other. The driver 240 may analyze the breathing pattern of the user by sensing the change in the impedance value of the fiber sensor 220.

The change in the impedance of each of the fiber sensors 220 occurs by a change in a distance between the user and each of the fiber sensors, even when each of the fiber sensors 220 does not come into contact with the user's body. Therefore, when the fiber sensors are utilized, the sleep of the user may be analyzed even when the user and the sensors do not come into contact with each other.

The fiber sensors 220a to 220d may be disposed on a central portion of the substrate and on both ends of the substrate. In one embodiment, four fiber sensors 220a to 220d may be arranged side by side in a longitudinal direction of the mat as shown in FIG. 3.

Because different signals are respectively generated from the fiber sensors, no matter where the user is located on the mat, at least one of the fiber sensors may sense the posture and the breathing pattern of the user.

In one example, each of the fiber sensors 220 may be formed in a stranded wire or a litz structure. Therefore, a sensing sensitivity may be increased compared to that in a single wire structure.

In one example, in order to induce the change in the impedance of each of the fiber sensors 220, a voltage must be applied to each of the fiber sensors. The driver 240 applies the voltage to each of the fiber sensors 220a to 220d and measures the change in the impedance of each of the fiber sensors 220a to 220d. Specifically, as shown in FIG. 6, the driver 240 may include an impedance matching circuit, a capacitance measuring circuit, a power supply, and an MCU. Additionally, the driver 240 may further include a communication unit for wireless communication with an external terminal.

In one example, the driver 240 may be detachable from the mat 200. To this end, the present disclosure may further include a connector 250 electrically connected to connecting electrodes 221a to 221d. The connector 250 may be formed to be detachable from the driver 240. The user may easily electrically connect the driver 240 to the connecting electrodes 221a to 221d by coupling the driver 240 to the connector 250. Based on the structure described above, because the user may wash only the remaining portions after removing the driver 240, the mat may be easily washed.

In one example, the human body sensing mat according to the present disclosure may include a temperature sensor, a humidity sensor, an illuminance sensor, an acoustic sensor, and the like. The driver 240 may provide sleep-related information based on information sensed by the sensors.

For example, the driver 240 may comprehensively analyze a temperature, a humidity, an illuminance, and a noise level around the mat, and output a sleep environment grade to the user. As the elements are more suitable for a sleeping environment, a higher grade may be output. The user may identify the sleep grade and take action to increase the sleep grade. Specifically, when the sleep grade is low due to low ambient humidity, the user may increase the sleep grade by operating a humidifier or the like to increase the ambient humidity.

In one example, the mat 200 according to the present disclosure may further include each of the connecting electrodes 221a to 221d for electrically connecting the driver 240 to each of the fiber sensors 220a to 220d. Specifically, each of the connecting electrodes 221a to 221d is disposed at an edge of the substrate to electrically connect each of the fiber sensors 220a to 220d to the driver 240. Because the individual connecting electrode is connected to each of the fiber sensors 220a to 220d, there may be the plurality of connecting electrodes.

The plurality of connecting electrodes may overlap each other at a specific point of the substrate. Specifically, as in an area A in FIG. 3, the number of overlapping connecting electrodes increases as the driver gets closer.

The connecting electrodes 221a to 221d may be made of the same material as that of the fiber sensors. In this case, the connecting electrodes 221a to 221d generate signals based on a distance from the specific object. Because it is difficult to distinguish the signals generated from the connecting electrodes 221a to 221d and the signals generated from the fiber sensors 220a to 220d respectively connected to the connecting electrodes from each other, each of the connecting electrodes may also be considered as a portion of each of the sensors.

It is not a problem to use each of the connecting electrodes as the portion of each of the sensors at a position where the connecting electrodes 221a to 221d do not overlap each other, but it is a problem to use each of the connecting electrodes as the portion of each of the sensors at a position where the connecting electrodes overlap each other.

For example, when the signal generated from the connecting electrodes 221a to 221d that are disposed adjacent to the driver 250 and are overlapping each other as in the area A in FIG. 3 is used for the sleep analysis, it is impossible to distinguish whether the signal is generated from the fiber sensor 220a located at a distal end of the substrate or the signal generated from the fiber sensor 220b located at the central portion of the substrate. When the user comes into contact with a position adjacent to the driver 240 during the sleep, an inaccurate sleep analysis result is calculated.

In order to prevent such problem, the mat according to the present disclosure may further include a shield for covering each of the connecting electrodes 221a to 221d and shielding an electromagnetic field.

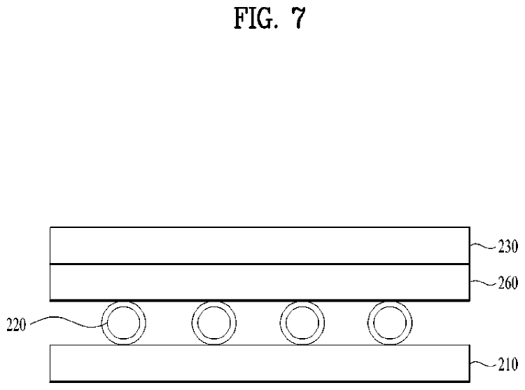

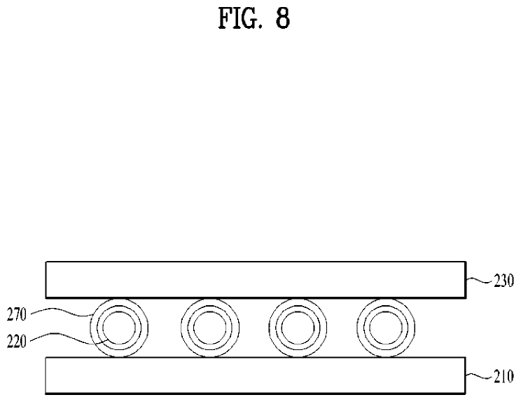

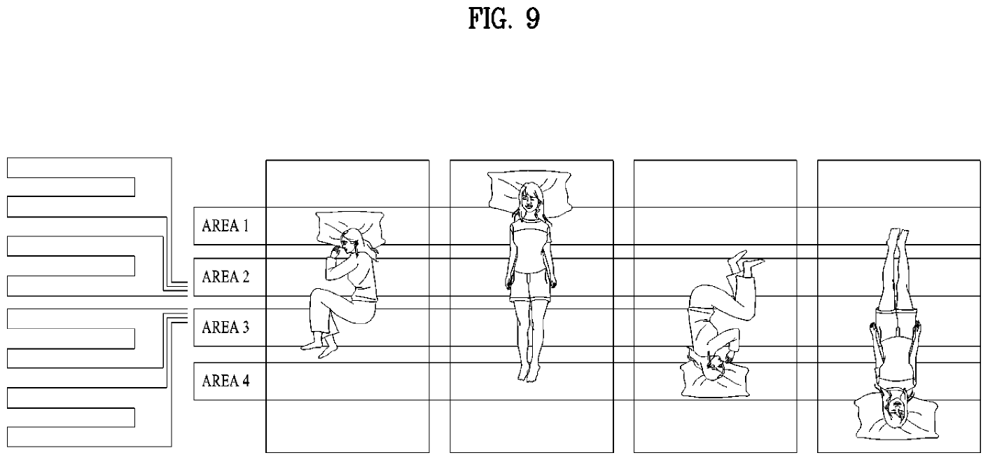

FIGS. 7 and 8 are cross-sectional views of a mat having a shield. FIG. 9 is a conceptual diagram illustrating an example of application of a human body sensing mat according to the present disclosure.

The shield blocks the electromagnetic field to prevent the change in the impedance of each of the connecting electrodes from occurring based on the change in the distance between each of the connecting electrodes and the specific object. When the shield is disposed at the edge of the substrate at which the connecting electrodes are disposed, the change in the impedance may be prevented from occurring at the connecting electrodes. Therefore, an inaccurate signal may be prevented from being generated at the position where the connecting electrodes overlap each other. In one embodiment, as shown in FIG. 7, the shield 260 may be formed in a sheet form. In another embodiment, as shown in FIG. 8, the shield 270 may be formed in a shape surrounding each of the connecting electrodes.

As described above, the present disclosure may utilize the shield to enable the accurate sleep analysis even when the user approaches the area where connecting electrodes overlap each other during the sleep.

Hereinafter, a specific example of application of the human body sensing mat according to the present disclosure will be described. In one embodiment, as shown in FIG. 9, when the fiber sensors are respectively disposed in four areas, no matter what posture the user sleeps in, a signal based on the posture change or the breathing pattern of the user may be generated from at least one of the four areas. Therefore, the present disclosure may analyze the sleep of the user regardless of the sleeping posture of the user.

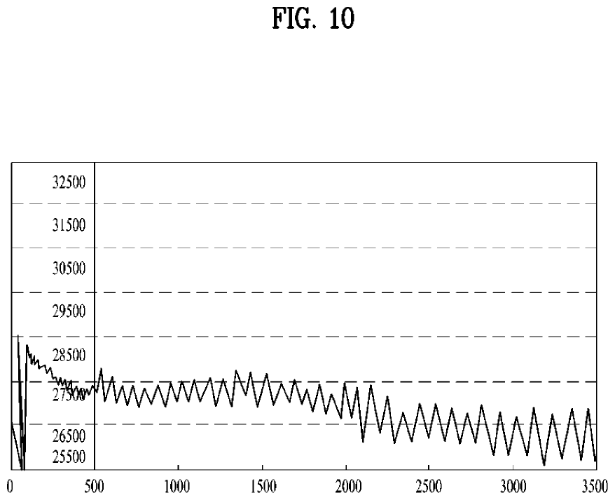

FIG. 10 is a graph showing a result of synthesizing signals generated from fiber sensors.

An x-axis in FIG. 10 represents a measurement time (seconds). Signals from 0 seconds to 500 seconds are signals of when the user has sleep apnoea, signals from 500 seconds to 2000 seconds are signals of when the user breathes normally, and signals from 2000 seconds to 3500 seconds are signals of when the user is breathing during the sleep. According to FIG. 10, it may be seen that the sleep apnoea, the normal breathing, and the breathing during the sleep of the user are clearly distinguished from each other.

In one example, the present disclosure may improve sleep analysis accuracy by synthesizing the signals generated from the fiber sensors placed at different positions on the mat. Hereinafter, a method for controlling a driver for improving the accuracy of the user's sleep analysis using the above-described human body sensing mat will be described.

The driver 240 may calculate posture information of the user based on the signal generated from each of the fiber sensors, and may output sleep information using the signal generated from each of the fiber sensors and the posture information.

Specifically, the driver 240 may calculate an area of the mat occupied by the user using the fiber sensors. Specifically, as in a first posture shown in FIG. 9, when the user occupies only areas 1 to 3, signals are generated only from fiber sensors respectively arranged in the areas 1 to 3. Based on such fact, the driver 240 may identify that the user is sleeping in a crouching posture. On the other hand, as in a second posture shown in FIG. 9, when the user occupies all of areas 1 to 4, signals are generated from all of the fiber sensors respectively arranged in the areas 1 to 4. Based on such fact, the driver 240 may identify that the user is sleeping with the body straight.

In one example, the driver 240 may output the sleep information to an output unit separately disposed on the mat or to a terminal capable of wireless communication with the driver 240.

In one example, the driver 240 may assign weights to the signals respectively generated from the fiber sensors based on the calculated posture information, and output the sleep information using the weighted signals.

Specifically, in analyzing the breathing pattern of the user, the driver 240 may assign a high weight to a signal generated from a fiber sensor disposed in a specific area. Specifically, the driver 240 may predict an area in which a chest of the user is located from the posture information, and assign the highest weight to a signal generated from a fiber sensor disposed in the area where the chest is located. This is because a body part where a change occurs the most when the user breathes is the chest.

For example, when the user is sleeping as in the first posture shown in FIG. 9, the driver 240 may predict that the chest of the user is placed in the area 2 from the posture information, and assign the highest weight to the signal generated from the fiber sensor disposed in the area 2 to analyze the breathing pattern of the user.

As described above, when the sleep of the user is analyzed based on the posture of the user, the sleep analysis accuracy may be improved.

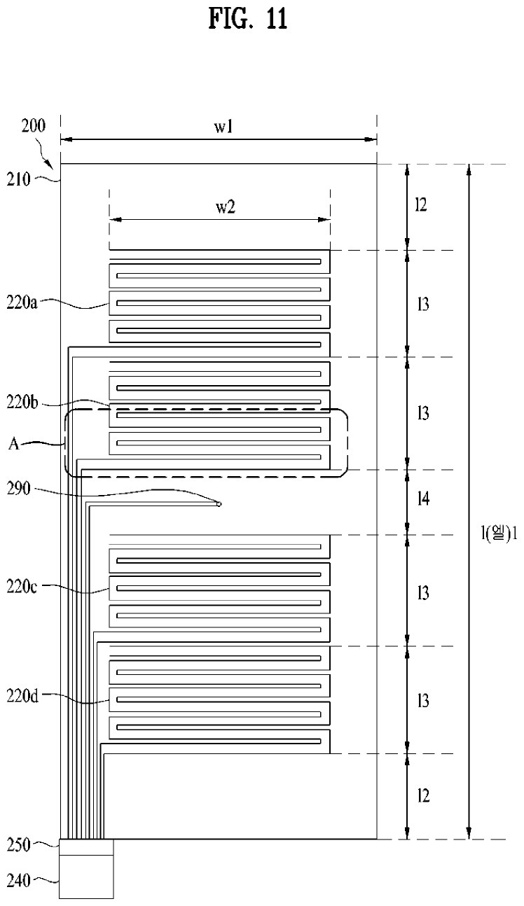

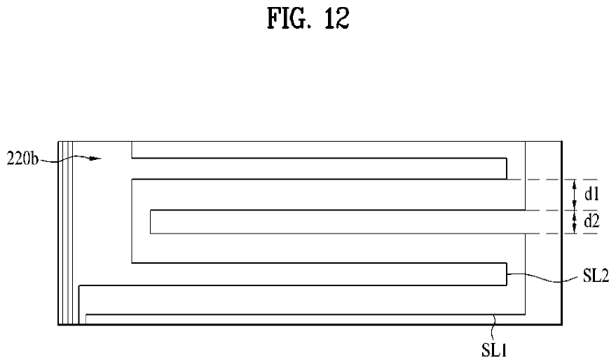

FIG. 11 is a diagram conceptually illustrating arrangement of the sensor arrays 220a, 220b, 220c, and 220d of the human body sensing mat 200 according to the present disclosure. FIG. 12 is an enlarged view of A in FIG. 11.

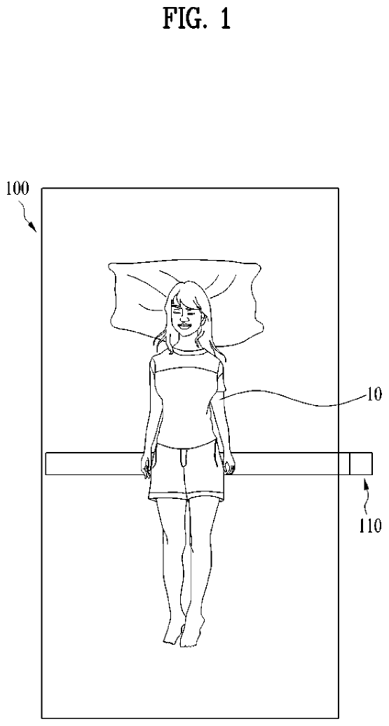

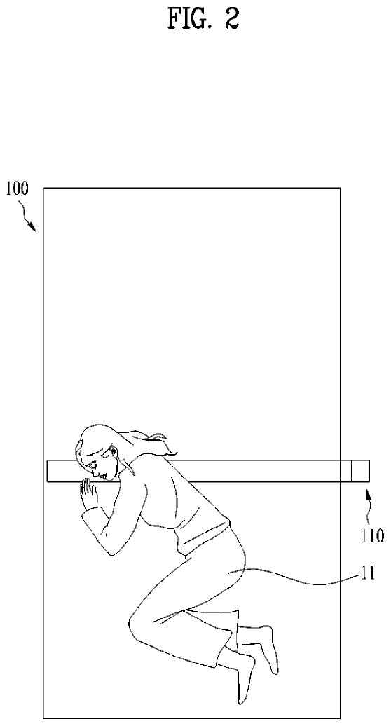

Referring to FIGS. 1 to 12, the substrate 210 may have a size corresponding to a size of a mattress of a single bed. For example, a width w1 of the substrate 210 may be about 1000 mm and a vertical dimension 11 thereof may be about 1950 mm.

In addition, the plurality of sensor arrays 220a, 220b, 220c, and 220d respectively corresponding to a plurality of areas or channels may be sequentially disposed in the central portion of the substrate 210 in a longitudinal direction. For example, the unit sensor arrays 220a, 220b, 220c, and 220d, each of which has a width w2 of 700 mm and a vertical dimension 13 of 313.5 mm, may be sequentially arranged in the longitudinal direction of the substrate 210 to respectively correspond to the four channels. In this regard, a distal sensor array among the unit sensor arrays 220a, 220b, 220c, and 220d may be disposed to be spaced apart from an end of the substrate 210 by a predetermined distance 12. In this regard, at least two (e.g., 220a and 220b or 220c and 220d) of the sensor arrays may be disposed adjacent to each other at one side or the other side of the substrate 210 corresponding to an upper body or a lower body of the user.

Referring to FIGS. 11 and 12, the sensor arrays 220a, 220b, 220c, and 220d may include a plurality of sensing lines SL1 and SL2 made of a conductive material arranged side by side in a first direction. In this regard, the first direction may be a width direction or a length direction of the substrate 210. However, the arrangement of the sensing lines shown in FIGS. 11 and 12 is illustrative, and arrangement of the sensing lines in a circular, oval, or triangular shape when necessary is also included in the scope of the present disclosure.

Referring to FIGS. 11 and 12, the pair of sensing lines SL1 and SL2 may be spaced apart from each other at a constant spacing and may be arranged in a zigzag form extending in the longitudinal direction of the substrate 210 from side to side. In this regard, the pair of sensing lines SL1 and SL2 may be connected to different electrodes. For example, a first sensing line SL1 and a second sensing line SL2 spaced apart from the first sensing line SL1 by the predetermined spacing and disposed in parallel with the first sensing line SL1 that are connected to the different electrodes may be disposed on the substrate 210 in the zigzag form.

Referring to FIG. 12, the plurality of sensing lines SL1 and SL2 may have a first spacing d1 between the adjacent sensing lines connected to the different electrodes greater than a second spacing d2 between adjacent sensing lines connected to the same electrode. In this regard, the first spacing d1 may be a distance between the first sensing line SL1 and the second sensing line SL2 closest to the first sensing line SL1, and the second spacing d2 may be a distance between a portion and another portion of the first sensing line SL1 or the second sensing line SL2. In this regard, the first and second spacings d1 and d2 may be distances in a direction perpendicular to the first direction. This is to minimize a measurement error because an interference between the sensing lines connected to the different electrodes is greater than an interference between the sensing lines connected to the same electrode.

When the spacing between the sensing lines is too great, a space efficiency and an accuracy of the measured impedance value are deteriorated. In addition, even when the spacing between the sensing lines is too small, the interference occurs between the adjacent sensing lines, resulting in an inaccurate measured impedance value. That is, in setting the first and second spacings d1 and d2, the space efficiency and the measurement accuracy are considered. For example, the first spacing d1 may be in a range from 25 mm to 30 mm, and the second spacing d2 may be in a range from 10 mm to 15 mm.

Referring to FIG. 11, the human body sensing mat 200 may further include a temperature sensor 290 disposed on the substrate 210 to measure a body temperature of the user. In this regard, the sensor array may include a first sensor array 220b disposed at one side of the substrate 210 to correspond to the upper body of the user and a second sensor array 220c disposed at the other side of the substrate 210 to correspond to the lower body of the user, and the temperature sensor 290 may be disposed at a location 14 between the first sensor array and the second sensor array. For example, a spacing 14 between the first and second sensor arrays may be about 196 mm. This is to calculate an accurate measurement value by placing the temperature sensor 290 in an area where the body temperature may be measured at all times despite the motion of the user during the sleep.

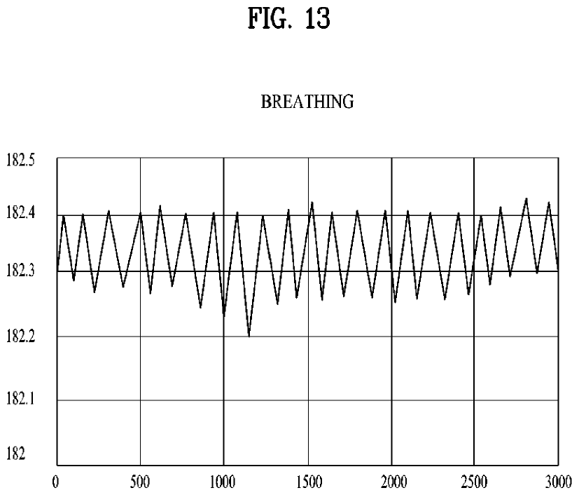

FIG. 13 is a diagram showing a result of sensing, by the human body sensing mat 200 in FIG. 11, breathing of a user.

Referring to FIGS. 10 to 13, when the sensing lines are arranged as shown in FIG. 11, it may be seen that a graph has a relatively regular and smooth shape than that in the case of FIG. 10.

That is, the human body sensing mat 200 according to an embodiment of the present disclosure may calculate a more accurate measurement result by minimizing noise caused by the interference between the sensing lines.

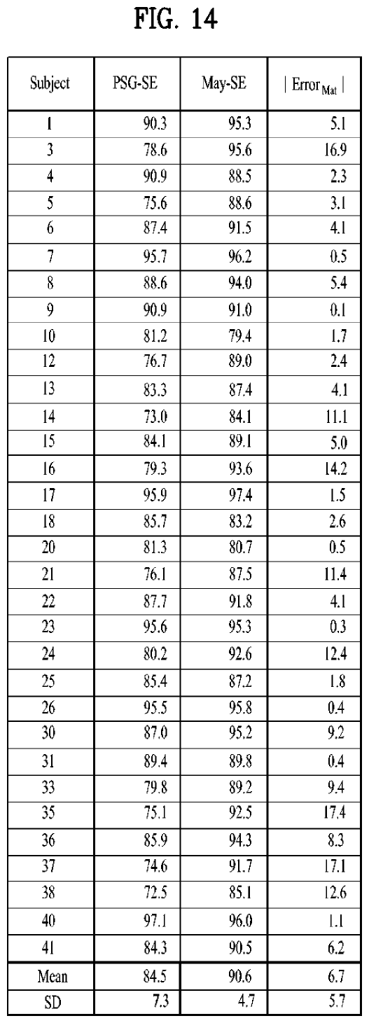

FIG. 14 is a diagram illustrating a table comparing measurement results of generally used polysomnography (PSG) equipment and the human body sensing mat 200 according to the present disclosure.

Referring to FIG. 14, numbers under PSG-SE are sleep efficiencies measured by the sleep analysis equipment, and numbers under Mat-SE are sleep efficiencies measured by the human body sensing mat 200.

Comparing the result measured by the sleep analysis equipment with the result measured by the human body sensing mat 200, it may be seen that a deviation Error mat between mean values Mean of the both results is about 6.7 percent, an accuracy of the sleep efficiency measured by the human body sensing mat 200 calculated as such is about 93.3 percent, and an error range SD is about 5.7 percent.

According to the present disclosure, because the fiber sensors are individually arranged at the central portion of the mat, the breathing, tossing and turning, and the like of the user may be sensed regardless of the posture of the user. Therefore, the present disclosure may analyze the motion and the bio-signal of the user regardless of the posture of the user.

In addition, according to the present disclosure, because the different signals are generated depending on the position of the mat, the sleeping posture of the user may be recognized. The present disclosure may improve the sleep analysis accuracy by utilizing the sleeping posture information of the user.

It is apparent to those skilled in the art that the present disclosure may be embodied in other specific forms without departing from the spirit and essential characteristics of the present disclosure.

In addition, the above detailed description should not be construed as restrictive in all respects, but should be considered as illustrative. The scope of the present disclosure should be determined by a reasonable interpretation of the appended claims, and all changes within the equivalent scope of the present disclosure are included in the scope of the present disclosure.

1. A human body sensing mat for sensing a motion of a human body, the human body sensing mat comprising:
a substrate;
a sensor array disposed on the substrate, the sensor array including a plurality of fiber sensors configured to generate signals based on a distance from a specific object; and
a driver configured to apply a voltage to the sensor array,
wherein the sensor array includes a plurality of sensing lines arranged in parallel with each other in a first direction,
wherein the plurality of sensing lines have a first spacing between adjacent sensing lines connected to different electrodes greater than a second spacing between adjacent sensing lines connected to a common electrode in a second direction.

2. The human body sensing mat of claim 1, wherein the first spacing and the second spacing are defined in a direction perpendicular to the first direction.

3. The human body sensing mat of claim 1, further comprising a temperature sensor disposed on the substrate and configured to measure a body temperature of a user on the human body sensing mat,
wherein the sensor array includes a first sensor array disposed at one side of the substrate and a second sensor array disposed at the other side of the substrate, wherein the first sensor array is located in the human body sensing mat to correspond to an upper body of the user and the second sensor array is located in the human body sensing mat to correspond to a lower body of the user,
wherein the temperature sensor is disposed between the first sensor array and the second sensor array.

4. The human body sensing mat of claim 1, wherein the plurality of sensing lines include a first sensing line, each and a second sensing line connected to different electrodes,
wherein the first sensing line and the second sensing line are spaced apart from each other at a predetermined spacing and arranged on the substrate in a zigzag form,
wherein the first spacing is a distance between the first sensing line and the second sensing line closest to the first sensing line, and
wherein the second spacing is a distance between a portion and another portion of the first sensing line or the second sensing line.

5. The human body sensing mat of claim 3, wherein the first spacing is in a range from 25 mm to 30 mm and a second spacing is in a range from 10 mm to 15 mm.

6. The human body sensing mat of claim 1, further comprising a plurality of connecting electrodes disposed at an respective ones of the substrate, wherein each of the plurality of connecting electrodes electrically connects each of the fiber sensors to the driver.

7. The human body sensing mat of claim 2, further comprising a shielding layer arranged to cover each of the connecting electrodes and to block an electromagnetic field.

8. The human body sensing mat of claim 3, wherein the shielding layer is formed at an edge of the substrate.

9. The human body sensing mat of claim 1, wherein the driver outputs sleep information of a user of the human body sensing mat using the signals respectively generated from the fiber sensors.

10. The human body sensing mat of claim 9, wherein each signal of the generated signals is an impedance change caused by a distance between the user and each fiber sensor.

11. The human body sensing mat of claim 9, wherein the driver calculates posture information of the user based on the signals respectively generated from the fiber sensors and outputs the sleep information using the signals respectively generated from the fiber sensors and the posture information.

12. The human body sensing mat of claim 11, wherein the driver assigns weights to the signals respectively generated from the fiber sensors based on the posture information and outputs the sleep information using the weighted signals.
